(19) United States
(12) Patent Application Publication
(10) Pub. No.: US 2012/0003164 A1
(43) Pub. Date: Jan. 5, 2012

(54) COSMETIC OR DERMATOLOGICAL COMPOSITION CONTAINING AN ACTIVE AGENT WHICH STIMULATES SYNTHESIS OF THE PROTEIN HSP 32 IN THE SKIN

(75) Inventors: Carine Nizard, Ivry S/Seine (FR); Marielle Moreau, Marcq (FR); Frédéric Bonte, Orleans (FR)

(73) Assignee: Parfums Christian Dior, Paris (FR)

(21) Appl. No.: 13/230,743

(22) Filed: Sep. 12, 2011

Related U.S. Application Data
(63) Continuation of application No. 10/869,703, filed on Jun. 15, 2004, now abandoned.
Application No. 10/869,703 is a division of application No. 09/869,692, filed on Aug. 27, 2001, now abandoned.

(30) Foreign Application Priority Data
Dec. 30, 1998 (FR) 9816641

(51) Int. Cl. A61K 8/97; A61Q 19/00; A61Q 19/08; A61Q 17/04

(52) U.S. Cl. 424/59

(57) ABSTRACT

A dermatological or cosmetological composition for an external topical administration, included together with pharmaceutically and/or cosmetologically acceptable excipients: at least one UVA-stabilizing and/or UVB-stabilizing screening agent, at least one compound capable of activating the endogenous synthesis of Heat Shock Protein (HSP) 32 or a functional peptide fragment of such a protein, and forskolin or any extract containing it.

This application is a divisional of U.S. patent application Ser. No. 09/869,692, filed Aug. 27, 2001, which claims priority under 35 U.S.C. §371 to PCT/FR99/03310 filed Dec. 29, 1999, which claims priority under FRANCE 98 16641 filed on Dec. 30, 1998.

### BACKGROUND OF THE INVENTION

1. Field

The present invention relates to compositions, in particular dermato-cosmetological compositions, which are useful in the field of photoprotection, and to cosmetic methods for treating skin exposed to solar radiation.

2. Description of the Related Art

Solar radiation, and mainly ultraviolet radiation, can cause harmful phenomena in the medium or long term. The solar energy reaching the ground is distributed, at wavelengths (λ) from 290 to 2 500 nm, for 50% in the infrared region (λ=800 to 2 500 nm), 40% in the visible region (λ=400 to 800 nm) and for 10% in the ultraviolet region, in which a distinction is made between the UVA region (λ=320-400 nm) and the UVB region (λ=290-320 nm)

Although immediate (UVA) or delayed (UVB) pigmentation constitutes a natural means of defense of the skin, exposure to ultraviolet radiation may cause actinic erythema, epidermal hyperplasia, cutaneous senescence (or solar elastosis) and even, in certain cases, may promote the onset of skin cancers.

Although the majority of the UVB radiation is absorbed by the horny layer, 10% reaches the dermis; the majority of the UVA radiation (and some of the visible radiation) crosses the epidermis and 20 to 30% reaches the dermis, where it may cause adverse changes in the skin cells.

It is accepted that UVA radiation causes the production of reactive oxygen species, in particular via the intracellular generation of H₂0₂ (Morlière et al., 1992)

The compounds have many target cells and cause various types of damage:

DNA: single- or double-strand cleavages, DNA-protein bridging,

proteins: singlet oxygen reacts with certain residues, including histidine and tryptophan,

membranes: peroxidation of polyethylenic fatty acids.

These adverse changes take place on all types of skin cells, in particular keratinocytes, melanocytes and fibroblasts, and are generally inflammatory manifestations or manifestations of actinic ageing (wrinkles).

It is also acknowledged that “HSPs” (“heat shock proteins”) have been demonstrated on cells, both eukaryotic and prokaryotic cells, subjected to physiological stress, in particular heat stress, both in vivo and in vitro. These cells react by expressing a set of proteins which vary in number and size depending on the target organism and the inducing stress (Maytin, E. D. (1995). Heat shock proteins and molecular chaperones: implications for adaptive responses in the skin. J. Invest. DFermato. 104, 448-454).

HSPs are classed in families according to their molecular weight. HSP 90, HSP 70, HSP 60 and HSP 30 have thus been distinguished. Many of the genes encoding the HSPs have been sequenced and their chromosomal location determined; however, little information is currently available regarding the transcriptional control of these molecules which are suspected of being among the cellular devices for protecting against a toxic environment.

Many factors may cause the induction of HSP5: high temperatures, heavy metals, viral infections, alcohol, growth factors and low temperatures (Simon, M. M., Reikerstorfer, A., Schwarz, A., Krone, C., Luger, T. Jaattela, A. and Scharz, T. (1995). Heat shock protein 70 overexpression affects the response to ultraviolet light in murine fribroblasts. J. Clin. Invest. 95, 926-933).

### SUMMARY

Advantageously, the compositions according to the invention contain at least one UVA-ray and/or UVB-ray screening agent. These screening agents are well known to those skilled in the art. Mention may be made, for example, of benzophenones, such as 2,2′,4,4′-tetrahydroxybenzophenone or Benzophenone-2 and 2-hydroxy-4′-methoxybenzophenone or Eusolex 4360®, which absorbs UVA and UVB radiation, cinnamate derivatives such as octyl p-methoxycinnamate or Parsol MCX®, which absorbs UVB radiation, dibenzoylmethane derivatives such as 4-tert-butyl-4′-methoxydibenzoylrnethane or Parsol 1789®, which absorbs UVA radiation, and paraaminobenzoic acid (Paba) esters, such as octyldimethyl PABA or Escalol 507®, which absorbs UVB radiation.

In this regard, it is important to note that the fibroblasts, which are major cells of the dermis giving the skin its tonicity, are the only skin cells in which it is particularly advantageous to induce the production of the protein HSP 32. It is thus particularly advantageous, in order to restore or conserve a good physiological condition of the skin, to stimulate the formation of this protein by the fibroblasts.

Consequently, the present invention also relates to the use of a compound capable of activating the endogenous synthesis of HSP 32, for the preparation of a cosmetic composition for protecting fibroblasts. Among the compounds described in the prior art which are capable of activating the synthesis of HSPs in general, mention should be made of patent application French Patent No. 2 757 863 which describes the use of biological substances of plant origin extracted from the fruit of plants with CAM metabolism.

### DETAILED DESCRIPTION

Among the compounds capable of promoting the endogenous production of HSP 32 by the fibroblasts, mention may be made of caffeic acid esters and derivatives thereof, in particular oraposide which has been described in documents WO 92/16544, French Patent No. 2 652 086, French Patent No. 2 708 851 and French Patent No. 2 699 818, and also PCOs (procyanidol oligomers) which have been described in documents European Patent No. 953 353, European Patent No. 955 051 and European Patent No. 397 914 and the article by J. Masquelier (“Oligomeres Procyanidoliques” PARFUMS, COSMETIQUES ET AR MES, no. 95, October 1990-November 1990 pages 89-97, XP000165236) and which may be extracted from grape and from green tea, for example, and also derivatives thereof.

Among the PCO derivatives which may be used, mention should also be made of the crosslinked PCOs as described in U.S. Pat. No. 5,780,060.

The compounds according to the present invention will preferably be used at concentrations of between 0.1% and 5% by weight of the composition and preferably at concentrations of between 0.2% and 1% by weight.

The compositions according to the present invention may comprise combinations of several “activating” compounds, as well as combinations with other advantageous components.

Among the preferred combinations, mention should be made more particularly of those which contain at least one compound chosen from:

forskolin or any extract containing it, in particular extracts of Plectiantrus barbatus,

tyrosine and its derivatives, in particular malyltyrosine,

ellagic acid and its derivatives or any extract containing them,

extracts of Centella asiatica, of Potentilla erecta and of Eriobotrya japonica,

soybean saponins and alfalfa saponins such as soyasapogenols,

isoflavones, in particular formononetin, daidzein and genistein or mixture thereof,

vitamin C and its derivatives, in particular vitamin C magnesium phosphate, tocopherol and its esters, in particular tocopheryl gentisate and tocopheryl phosphate,

18-β-glycyrrhetinic acid, —extracts of Azadiracta indica,

curcuminoids, in particular a curcumin.

It is advantageous to note that the compositions according to the present invention can also contain heat shock proteins, in particular the protein HSP 32 itself or an active fragment thereof.

Preferably, the compositions according to the present invention will be in a form which is suitable for topical cutaneous administration.

These compositions may especially be in the form of solutions, suspensions, lotions, milks, gels, creams, 01W or W/0 emulsions or multiple emulsions, sticks or powders, suitable for application to the skin, the lips and/or the hair.

They comprise the excipients required for this formulation, such as solvents, diluents, thickeners, ionic or nonionic surfactants, in particular sucroesters, preserving agents, antioxidants, colorants, fragrances or, when they are packaged as aerosols, propellant gases.

The compositions may also contain softeners, moisturizers, anti-inflammatory agents, anti-wrinkle agents, in particular agents promoting the synthesis of glycosamino-glycan (GAG), or tanning activators.

Advantageously, the compositions according to the invention contain a free-radical scavenger, for example α-tocopherol or its esters.

According to one of the embodiments of the invention, the composition also contains at least one photoprotective agent, preferably chosen from the group consisting of physical sunblocks and sunscreens.

Sunscreens are molecules capable of absorbing radiation within a more or less broad region of the solar spectrum. They may belong to various classes; mention may be made, in a non-limiting manner, of paraaminobenzoic acid and its derivatives, cinnamic acid esters, salicylic acid derivatives or benzylidenecamphor derivatives, benzimidazoles and benzophenone derivatives.

The physical sunblocks which may be used are, in particular, titanium oxide, zinc oxide, mica derivatives and talc.

The presence of physical sunblocks or sunscreens in the composition will make it possible to improve the protection against solar radiation of the body surface onto which it is applied.

A subject of the invention is also the use of at least one compound chosen from the group consisting of PCOs and derivatives thereof, caffeic acid esters and derivatives thereof and mixtures of these compounds, for the preparation of a composition intended to activate the endogenous synthesis of HSP 32 or a functional peptide fragment of such a protein.

The preferential aspects stated above for the composition per se are also valid for the composition prepared and intended to activate the endogenous synthesis of HSP 32 or a functional peptide fragment of such a protein according to this use. In particular, the use according to the invention is characterized in that the composition contains pharmaceutically and/or cosmetologically acceptable excipients.

Another subject of the invention is a cosmetic method for treating the skin and integuments in order to protect them against the harmful effects of radiation, in particular ultraviolet radiation, characterized in that an effective amount of a composition as described above is applied locally, before or at the time of exposure to radiation, in particular ultraviolet radiation, for example solar radiation.

More particularly, the above method is intended to combat the formation of solar erythema, solar allergies or solar elastosis and to prevent or delay the appearance of wrinkles caused by the harmful effects of ultraviolet radiation.

Finally, according to another of its aspects, the invention comprises the use of these compositions as medicinal products, in particular in dermatology.

A subject of the invention is also the application, as a cosmetic product, of the heat shock protein HSP 32.

In the examples which follow, the protective effect of PCOs by means of inducing HSP 32 will be demonstrated as a function of an administration or otherwise of UVA.

### Example 1

The following examples were carried out using fibroblast cell cultures which are or are not subjected to the treatment with PCOs and then on which, after UVA radiation, the induction of HSP 32 is assayed by means known to those skilled in the art.

These means in particular comprise the use of an antiHSP 32 primary antibody, commercially available from the company TEBU, in a technique known as immunodetection.

The results obtained are collated in the table below.

### Effect of PCO on the Expression of the Protein HSP 32 with Our without UVA (Western Blot)

| | CONTROL | | | | PCO 25 μg/ml | | | | PCO 50 μg/ml | | | |
|---|---|---|---|---|---|---|---|---|---|---|---|---|
| | UV− | | UV+ | | UV− | | UV+ | | UV− | | UV+ | |
| Volume density | 95 | 832 | 125 | 208 | 140 | 935 | 123 | 265 | 163 | 328 | 195 | 552 |

| | | | | | | |
|---|---|---|---|---|---|---|
| Effect/control | 100% | 131% | 147% | 128% | 170% | 204% |
| UV− | | | | | | |

It is found that the UVA naturally induces the synthesis of HSP 32 (protein quantified by Western blot) but this synthesis remains moderate. The addition of PCO stimulates the induction of the HSP 32 molecules more strongly than UVA alone, in particular when the PCOs are used at 50 μg/ml.

Treating the cells with PCOs followed by TJVA irradiation leads to a massive stimulation of the production of HSP since it may be up to 204% when the PCOs are used at 50 JAg/mi.

The protective effect of these PCOs is thus clearly demonstrated, both with and without irradiation. Thus, the compositions may be used preventively and/or curatively, preferably in combination with UVA stabilizing and/or UVE-stabilizing screening agents.

### Example 2

### Cosmetic Composition

### Antisun Bodycare Cream of SPF 15

| | |
|---|---|
| PCO from grape seed | 0.5 |
| Ceramide 3 | 0.12 |
| Glyercol | 2 |
| Octyl methoxycinnamate | 7.5 |
| Parsol 1789 ® | 2 |
| Tocoheryl acetate | 0.2 |
| Fragrance emulsion excipient, qs | 100 |

### Tanning and Protective Facial Sunscreen

| | |
|---|---|
| PCO from green tea | 0.5 |
| Extract of Plecthantrus barbatus | 0.05 |
| Tyrosine | 1 |
| Hyaluronic acid | 0.2 |
| Eusolex 4360 ® | 8 |
| Glycerol | 3 |
| α-Tocopheryl acetate | 0.2 |
| Fragrance excipient, qs | 100 |

### Anti-Wrinkle Care Cream

| | |
|---|---|
| Extract of Plecthantrus barbatus | 0.01 |
| Arginine | 0.2 |
| Ellagic acid | 0.2 |
| PCO from green tea | 0.4 |
| Extract of Centella asiatica | 0.5 |
| Octyl methoxycinnamate | 2 |
| Excipient, qs | 100 |

What is claimed is:

1. A dermatological or cosmetological composition for an external topical administration, comprising together with pharmaceutically and/or cosmetologically acceptable excipients:
at least one UVA-stabilizing and/or UVB-stabilizing screening agent,
at least one compound capable of activating the endogenous synthesis of Heat Shock Protein (HSP) 32 or a functional peptide fragment of such a protein, and
forskolin or any extract containing it.

2. The composition according to claim 1, wherein the extract containing forskolin is selected among extracts of Plectranthus barbatus.

3. The composition according to claim 1, wherein the compound capable of activating the endogenous synthesis of HSP 32 is selected from the group consisting of Procyanidol Oligomers (PCOs) and derivatives thereof, caffeic acid esters and derivatives thereof and mixtures of these compounds.

4. The composition according to claim 2, wherein the Procyanidol Oligomer (PCO) derivative is a crosslinked PCO.

5. The composition according to claim 2, wherein the Procyanidol Oligomer (PCO) is a PCO from grape seed or a PCO from green tea.

6. The composition according to claim 1, wherein said compound is present in a concentration of between 0.1% and 5% w/w in the composition.

7. The composition according to claim 6, wherein said compound is present in a concentration of between 0.2% and 1% w/w in the composition.

8. The composition according to claim 1, wherein the composition also contains at least one other photo-protective agent.

9. The composition according to claim 1, wherein the composition further contains at least one compound chosen from the group consisting of physical sunblocks, sunscreens and free-radical scavengers.

10. The composition according to claim 1, wherein the composition further contains the Heat Shock Protein (HSP) 32 or an active fragment thereof.

11. A cosmetic method for treating the skin or integuments in order to protect them against the harmful effects of radiation, comprising the local application to said skin or integuments of an effective amount of at least one composition according to claim 1, before or at the time of exposure to said radiation.

12. The cosmetic method according to claim 11, wherein the radiation is ultraviolet radiation.

13. The method according to claim 11, for combating the formation of solar erythema, solar allergies or solar elastosis.

14. The method according to claim 11, for preventing or delaying actinic ageing of the skin.

15. The method according to claim 14, for preventing or delaying the appearance of wrinkles caused by the harmful effects of ultraviolet radiation.

16. Use of the Heat Shock Protein (HSP) 32, as a cosmetic product.

17. The composition according to claim 1, wherein the composition further contains at least one component chosen from:
forskolin or any extract containing it, in particular extracts of Plecthantrus barbatus;
tyrosine and its derivatives, in particular malyltyrosine;
ellagic acid and its derivatives or any extract containing them;
extracts of Centella asiatica, of Potentilla erecta and of Eriobotrya japonica;
soybean saponins and alfalfa saponins such as soyasapogenols;
isoflavones, in particular formononetin, daidzein and genistein or mixture thereof;
vitamin C and its derivatives, in particular vitamin C magnesium phosphate, tocopherol and its esters, in particular tocopheryl gentisate and tocopheryl phosphate;
18-β-glycyrrhetinic acid;
extracts of Azadiracta indica;
curcuminoids, in particular a curcumin.

18. The composition according to claim 1 in combination with at least one component chosen from:
forskolin or any extract containing it, in particular extracts of Plecthantrus barbatus;
tyrosine and its derivatives, in particular malyltyrosine, with the exception of L-DOPA (or “3-hydroxy-L-tyrosine”);
ellagic acid and its derivatives or any extract containing them;
extracts of Centella asiatica, of Potentilla erecta and of Eriobotrya japonica;
soybean saponins and alfalfa saponins such as soyasapogenols;
isoflavones, in particular formononetin, daidzein and genistein or mixture thereof;
vitamin C and its derivatives, in particular vitamin C magnesium phosphate, tocopherol and its esters, in particular tocopheryl gentisate and tocopheryl phosphate;
18-β-glycyrrhetinic acid;
extracts of Azadiracta indica;
curcuminoids, in particular a curcumin;
with pharmaceutically and/or cosmetically acceptable excipients, for the preparation of a composition intended cosmetological use.
